(19) United States
(12) Patent Application Publication
(10) Pub. No.: US 2025/0116664 A1
(43) Pub. Date: Apr. 10, 2025

(54) Monoclonal Antibody, Colloidal Gold Test Strip For Stichopus Oligopeptide

(71) Applicant: DALIAN DEEP BLUE PEPTIDE TECHNOLOGY RESEARCH AND DEVELOPMENT CO., LTD., Shenyang (CN)

(72) Inventors: Weiyang BAO, Shenyang (CN); Zuzhe WANG, Shenyang (CN); Aihua ZUO, Shenyang (CN); Pu MA, Shenyang (CN); Tianli SUN, Shenyang (CN); Linying ZHAO, Shenyang (CN)

(21) Appl. No.: 18/918,121

(22) Filed: Oct. 17, 2024

Related U.S. Application Data
(63) Continuation-in-part of application No. 17/418,268, filed on Jun. 25, 2021.

(51) Int. Cl. G01N 33/543; C07K 16/44; G01N 33/53; G01N 33/553

(52) U.S. Cl. CPC G01N 33/54388; C07K 16/44; G01N 33/553; C07K 2317/24; G01N 33/5308

(57) ABSTRACT

The invention belongs to the field of biotechnology, in particular, relates to peptide fragment, monoclonal antibody, colloidal gold test strip and detection method thereof used for detection of stichopus oligopeptide. Sequences of the peptide fragments are SEQ ID NO: 1 and SEQ ID NO: 3. After identification, the peptides SEQ ID NO: 1 and SEQ ID NO: 3 are newly discovered oligopeptides, which are exclusive peptide fragments of Stichopus oligopeptide.

### CROSS REFERENCES TO RELATED APPLICATIONS

This application is a Continuation-In-Part (CIP) of U.S. patent application Ser. No. 17/418,268 filed on Jun. 25, 2021, which is a National Phase Filing of PCT/CN2020/113918 filed on Sep. 8, 2020, which claims priority to CN202010038879.4 filed on Jan. 14, 2020, the contents of which are incorporated herein by reference in their entirety.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: one 16,000 Byte XML file named “21A0744US.xml,” dated Sep. 5, 2024.

### TECHNICAL FIELD

The invention belongs to the field of biotechnology, in particular, relates to peptide fragment, monoclonal antibody, colloidal gold test strip and detection method thereof used for detection of stichopus oligopeptide.

### BACKGROUND

Holothurian belongs to Echinodermata, holothurian class and Scutellara animals. It is a rare nutritional and health food with high protein, low fat, low sugar and extremely low cholesterol in the world, and has extremely high nutritional value and medicinal value. Compared with different varieties of holothurians, the contents of protein, polysaccharide and minerals are quite different. There are about 1200 species of holothurians in the world, but most of them have no edible value. According to statistics, there are about 40 kinds of edible holothurians in the world, while there are only about 20 kinds of edible holothurians in China, and only a few kinds of holothurians have high commercial value.

Stichopus is a very important economic aquatic animal in China, which is not only rich in nutrition, but also has reasonable nutrient composition. It is a kind of seafood which is deeply loved by people and has high nutritional value. The stichopus oligopeptide is obtained from sea cucumbers as raw materials by enzymatic hydrolysis with biological enzymes, which has the advantages of small molecular weight, easy absorption and high bioavailability.

In recent years, a variety of oligopeptide products have appeared on the market, all of which are white or yellowish powder. It is impossible to identify the source of oligopeptides with naked eyes or rapid and effective methods. Due to the lack of effective methods to identify stichopus oligopeptide at present, many unscrupulous merchants use sea eggplant, Panax ginseng and other raw materials to prepare oligopeptides that are shoddy, and even use peptides prepared by other high-protein animals and plants as stichopus oligopeptide to deceive consumers. Therefore, an efficient and accurate method for identifying stichopus oligopeptide is needed.

### SUMMARY

In response to the shortcomings and requirements in the above fields, the present invention provides peptide fragment, monoclonal antibody, colloidal gold test strip and detection method thereof used for detecting stichopus oligopeptide.

According to the first embodiment, the present application provides a monoclonal antibody against KIVPGVPD (SEQ ID NO. 1) or GRDGDQGPV (SEQ ID NO. 3), wherein the monoclonal antibody comprises: (a) a heavy chain variable region with amino acid sequence of SEQ ID NO: 5, and a light chain variable region with amino acid sequence of SEQ ID NO: 6; or (b) a heavy chain variable region with amino acid sequence of SEQ ID NO: 7, and a light chain variable region with amino acid sequence of SEQ ID NO: 8; or (c) a light chain variable region with amino acid sequence of SEQ ID NO: 9, and a heavy chain variable region with amino acid sequence of SEQ ID NO: 10.

According to the second embodiment, the present application provides a colloidal gold test strip which comprises a sample pad, a gold label pad coated with the above-said monoclonal antibody, an NC membrane, an absorbent pad and a PVC bottom plate.

In particular, the sample pad, the gold label pad, the NC film and the absorbent pad are sequentially arranged on the PVC bottom plate according to the sample chromatography direction.

Preferably, the NC membrane sequentially comprises a detection line and a quality control line according to the direction of sample chromatography, wherein the detection line is coated with a conjugate of a peptide fragment and bovine serum albumin, and the quality control line is coated with goat anti-mouse secondary antibody.

Further, the monoclonal antibody labeled with colloidal gold is prepared according to the following method:

Taking 100 ml of 0.01% chloroauric acid solution and heat it, adding 2 ml of 1% trisodium citrate after boiling, and continuing to boil for 5 minutes after the solution turns to wine red. After cooling to room temperature, making up to 100 ml with ultrapure water to obtain a colloidal gold solution with a gold particle diameter of about 10 nm. Adjusting the pH value of the obtained 100 ml colloidal gold solution to 8.2 with 20 mol/L borate buffer, stirring the colloidal gold solution and slowly adding 45 μg of monoclonal antibody per ml of colloidal gold solution, reacting for 30 minutes, adding 10% BSA in volume fraction to a final concentration of 1% in volume fraction, and stirring for 10 minutes, centrifuging at 45,000 rpm for 30 min at 4° C., discarding the supernatant and the resulting precipitate is the purified colloidal gold-labeled monoclonal antibody.

The present invention provides a peptide fragment, monoclonal antibody, colloidal gold test strip and detection method thereof used for detecting stichopus oligopeptide. The principle is: After the sample is dropped onto the sample pad, it swims in the direction of the absorbent pad, the colloidal gold-labeled monoclonal antibody (gold-labeled antibody) against KIVPGVPD (SEQ ID NO. 1) or GRDGDQGPV (SEQ ID NO. 3) peptide on the gold label pad is dissolved. If the sample is not stichopus oligopeptide, then when the gold-labeled antibody reaches the detection line on the NC membrane, the gold-labeled antibody is captured by the antigen-BSA conjugate coated on the detection line, and deposited to turn the detection line red. The excess gold-labeled antibody continues to spread forward and is captured by the goat anti-mouse secondary antibody on the quality control line, making the quality control line red. If the sample is stichopus oligopeptide, the gold-labeled antibody combines with the peptide fragment KIVPGVPD (SEQ ID NO. 1) or GRDGDQGPV (SEQ ID NO. 3) and swims forward, crosses the detection line, and is captured by goat anti-mouse secondary antibody on the quality control line, making the quality control line turn red while the detection line does not change color. Only when the quality control line is red, the detection result is effective. The method of the invention has high accuracy, with a detection sensitivity of 20 g/ml, good specificity, easy observation of results, and can realize rapid screening of large quantities of samples.

### BRIEF DESCRIPTION OF DRAWINGS

FIG. 1 is a structural schematic diagram of a colloidal gold test strip prepared by using specific peptide fragments of the present invention.

FIG. 2 is a schematic diagram of the detection results of the colloidal gold test strip prepared by using specific peptide fragments of the present invention.

### DESCRIPTION OF THE EMBODIMENTS

The invention is described in detail in combination with the drawings and specific embodiments, but it should not be understood as a limitation of the invention. Unless otherwise specified, the technical means used in the following embodiments are conventional means well known to those skilled in the art, and the materials and reagents used in the following examples can be obtained from commercial sources unless otherwise specified.

### Example 1

### Sequence Source and Alignment Information of Specific Peptide Fragments (Antigen)

### 1. Sources of Stichopus-Specific Oligopeptide KIVPGVPD

Stichopus-specific oligopeptide KIVPGVPD, whose amino acid sequence is as shown in SEQ ID NO. 1, is derived from protein 2 alpha fibrillar collagen, whose Accesion number on NCBI is PIK60694, and whose amino acid sequence is as shown in SEQ ID NO. 2. After NCBI blast comparison, the peptide fragment was not consistent with any other species, and it is further identified as a new oligopeptide by online database BIOPEP and EROP-Moscow.

### 2. Sources of Stichopus-Specific Oligopeptide GRDGDQGPV

Stichopus-specific oligopeptide GRDGDQGPV has an amino acid sequence as shown in SEQ ID NO. 3 and is derived from protein alpha-2 collagen. Its Accesion number on NCBI is PIK60696, and its amino acid sequence is shown in SEQ ID NO. 4. After NCBI blast comparison, the peptide fragment was not consistent with any other species, and it is further identified as a new oligopeptide by online database BIOPEP and EROP-Moscow.

### Example 2

### Preparation of Colloidal Gold Test Strip

### 1. Colloidal Gold Test Strip Prepared by Peptide Fragment KIVPGVPD

10 mg of BSA was dissolved in 0.01 mol/L PBS (pH 7.4) (solution 1). 4 mg of purified KIVPGVPD peptide fragment was dissolved in 0.01 mol/L PBS (pH 7.4) (solution 2). 4 mg of 1-(3-dimethylaminopropyl)-3-ethylcarbodiimide hydrochloride (EDC) was dissolved in 0.5 ml of 0.01 mol/L PBS (pH 7.4) (solution 3). Solution 2 was added into solution 1, mixed well in ice bath for 30 min, centrifuged to remove precipitation, the supernatant was added dropwise into solution 3, with continued ice bath for 15 min, and stirred at room temperature for 5 min. Dialysis was performed in 0.01 mol/L PBS (pH7.4) for 24h, during this period, the dialysate was changed for twice, and centrifugation was carried out to remove precipitation to obtain the conjugate of the peptide fragment KIVPGVPD and bovine serum albumin. BALB/C mice was immunized with KIVPGVPD-BSA; the spleen lymphocytes of immunized mice and myeloma cell (SP2/0) were fused under the promotion of PEG, and the hybridoma cell line stably secreting KIVPGVPD-BSA monoclonal antibody was screened. The hybridoma cell line was grown in increments, and the obtained culture solution was purified by the caprylic acid-saturated ammonium sulfate method to obtain monoclonal antibodies, which was frozen and stored at −20° C. for later use. One monoclonal antibody has the sequence of:

Heavy Chain (VH + CH):
(SEQ ID NO. 5)
QVQLVQSGAEVKKPGSSVKVSCKASGYAFTSYTMHWVKQAPGQGLEWIG
YINPSRGYTNYNQKFKDKATLTTDKSSSTAYMQLSSLTSEDSAVYYCGY
ISGIYPGSGVRDGYDYWGQGTLVTVSSASTKGPSVFPLAPSSKSTSGGT
AAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLG
GPSVFLFPPKPKDTLMISRTPASVSGSPGQSVEKTVAWKADSSPVKAGV
ETTTPSKQSNNKYAASSYLSLTPEQWKSHRSYSCQVTHEGSTVEKTVAP
TECS,
and
Light Chain (VL + CL):
(SEQ ID NO. 6)
DIVMTQSPSFLSASVGDRVTITCRASQSVSSYLAWYQQKPGKAPKLLIY
GSSNRASGVPDRFSGSGSGTDFTLTISSVEAEDVAVYYCKASQDVVLTS
ASNRFSQQYYSTPWTFGQGTKVEIKRTVAAPSVFIFPPSDEQLKSGTAS
VVCLLNNFYPRDISVKWKIDGSERQNGVLNSWTDQDSKDSTYSMSSTLT
LTKADYEKHKVYACEVTHQGLSSPVTKSFNRGEC.

An Erlenmeyer flask containing 100 ml of 0.01% chloroauric acid solution was put on a magnetic stirrer to heat and stir. 2 ml of 1% trisodium citrate was added quickly after boiling, and continued to boil for 5 minutes after the solution turned into wine red. After cooling to room temperature, made up to 100 ml with ultrapure water to obtain a colloidal gold solution with a gold particle diameter of about 10 nm. The pH value of the obtained 100 ml colloidal gold solution was adjusted to 8.2 with 20 mol/L borate buffer, 45 μg of monoclonal antibody KIVPGVPD-BSA per ml of colloidal gold solution was added slowly while stirring on a magnetic stirrer, and reacted for 30 minutes. 10% BSA was added to a final concentration of 1%, and gently stirred for 10 minutes. Centrifuged at 45,000 rpm for 30 min at 4° C., discarded the supernatant and the resulting precipitate was the purified colloidal gold-labeled monoclonal antibody. This colloidal gold-labeled monoclonal antibody was sprayed on the gold label pad, the KIVPGVPD-BSA conjugated on the NC film was sprayed as a test line, goat anti-mouse IgG on NC membrane was sprayed as a quality control line, then the sample pad (glass fiber), gold label pad (glass fiber), NC film (including quality control line and test line) and the absorbent pad were adhered to a PVC bottom plate in sequence.

### 2. Colloidal Gold Test Strip Prepared by Peptide GRDGDQGPV

10 mg of BSA was dissolved in 0.01 mol/L PBS (pH 7.4) (solution 1). 4 mg of purified GRDGDQGPV peptide fragment was dissolved in 0.01 mol/L PBS (pH 7.4) (solution 2). 4 mg of 1-(3-dimethylaminopropyl)-3-ethylcarbodiimide hydrochloride (EDC) was dissolved in 0.5 ml of 0.01 mol/L PBS (pH 7.4) (solution 3). Solution 2 was added to solution 1, mixed well in ice bath for 30 min, centrifuged to remove precipitation, then it was added dropwise into solution 3, with continued ice bath for 15 min, and stirred at room temperature for 5 min. Dialysis was performed in 0.01 mol/L PBS (pH7.4) for 24h, during this period, the dialysate was changed for twice, and centrifugation was carried out to remove precipitation to obtain the conjugate of the peptide fragment GRDGDQGPV and bovine serum albumin. BALB/C mice was immunized with GRDGDQGPV-BSA; the spleen lymphocytes of immunized mice and myeloma cell (SP2/0) were fused under the promotion of PEG, and the hybridoma cell line stably secreting GRDGDQGPV-BSA monoclonal antibody was screened. The hybridoma cell line was grown in increments, and the obtained culture solution was purified by the caprylic acid-saturated ammonium sulfate method to obtain monoclonal antibodies, which was frozen and stored at −20° C. for later use. One monoclonal antibody has the sequence of:

Heavy Chain (VH + CH):
(SEQ ID NO. 7)
QVQLVQSGAEVKKPGSSVKVSCKASGYAFTSYTMH
WVKQAPGQGLEWIGYINPSRGYTNYNQKFKDKATLTTDKSSSTAYMQLS
SLTSEDSAVYYCRASQDISNYLNYASRLSGRYFDYWGQGTLVTVSSAST
KGPSVFPLAPSSKSTSGGTAAALGCLVKDYFPEPVTVSWNSGALTSGVH
TFPAVLQSSGLYSLSSVVTVPSSSLGTQTYICNVNHKPSNTKVDKKVEP
KSCDKTHTCPPCPAPELLGGPSVFLFPPKPKDTLMISRTPASVSGSPGQ
SVEKTVAWKADSSPVKAGVETTTPSKQSNNKYAASSYLSLTPEQWKSHR
SYSCQVTHEGSTVEKTVAPTECS,
and
Light Chain (VL + CL):
(SEQ ID NO. 8)
DIVMTQSPSFLSASVGDRVTITCRASQSVSSYLA
WYQQKPGKAPKLLIYGSSNRASGVPDRFSGSGSGTDFTLTISSVEAEDV
AVYYCKASQDVVLTSASNRFSQQYYSTPWTFGQGTKVEIKRTVAAPSVF
IFPPSDEQLKSGTASVVCLLNNFYPRDISVKWKIDGSERQNGVLNSWTD
QDSKDSTYSMSSTLTLTKADYEKHKVYACEVTHQGLSSPVTKSFNRGE
C.

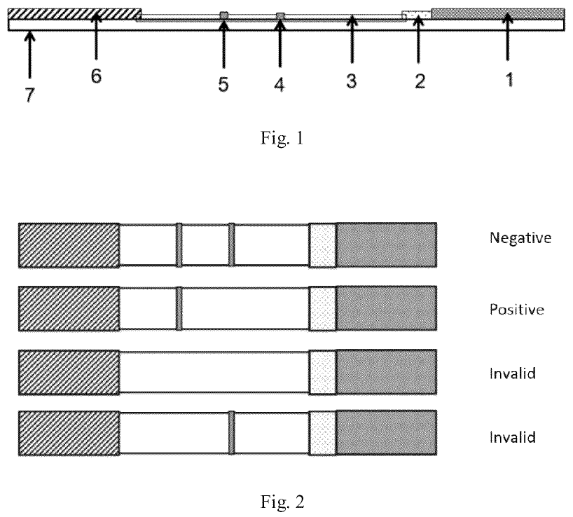

An Erlenmeyer flask containing 100 ml of 0.01% chloroauric acid solution was put on a magnetic stirrer to heat and stir. 2 ml of 1% trisodium citrate was added quickly after boiling, and continued to boil for 5 minutes after the solution turns to wine red. After cooling to room temperature, made up to 100 ml with ultrapure water to obtain a colloidal gold solution with a gold particle diameter of about 10 nm. The pH value of the obtained 100 ml colloidal gold solution was adjusted to 8.2 with 20 mol/L borate buffer, 45 μg of monoclonal antibody GRDGDQGPV-BSA per ml of colloidal gold solution was added slowly while stirring on a magnetic stirrer, and reacted for 30 minutes. 10% BSA was added in gold standard solution to a final concentration of 1%, and gently stirred for 10 minutes. The solution was centrifuged at 45,000 rpm for 30 min at 4° C., the supernatant was discarded and the resulting precipitate was the purified colloidal gold-labeled monoclonal antibody. This colloidal gold-labeled monoclonal antibody was sprayed on the gold label pad, the GRDGDQGPV-BSA conjugated on the NC film was sprayed as a test line, goat anti-mouse IgG on NC membrane was sprayed as a quality control line, then the sample pad (glass fiber), gold label conjugate pad (glass fiber), NC film (including quality control line and test line) and the absorbent pad were adhered to a PVC bottom plate in sequence. FIG. 1 shows the structure of the resulted colloidal gold test strip of the present example, wherein: 1. Sample pad; 2. gold label pad; 3. NC membrane; 4. detection line; 5. quality control line; 6. absorbent pad; 7. PVC bottom plate.

### Example 3

### Detecting Stichopus Oligopeptide by Colloidal Gold Immunoassay

S1. The sample was dissolved with distilled water to 1 mg/ml, and 3 drops were added to the sample pad of the colloidal gold test strip prepared in Example 2.

S2. After the quality control line developed color, observed whether the detection line changes from colorless to red; if the detection line turned red, the sample to be tested was not stichopus oligopeptide; if the detection line did not change color, the sample to be tested was stichopus oligopeptide.

### Example 4

### Sensitivity Detection of Colloidal Gold Test Strip

According to the method described in Example 3, stichopus oligopeptide with 8 gradients, respectively 0 μg/ml, 5 μg/ml, 10 μg/ml, 20 μg/ml, 40 μg/ml, 80 μg/ml, 100 μg/ml, and 1000 μg/ml were detected with the detection strip described in Example 2, and the detection line was observed with naked eyes three times. The test results are as shown in FIG. 2. It was found that a deep red band appears on the detection line when the concentration of stichopus oligopeptide was 0 μg/ml. With the increase of the concentration, the color of the detection line gradually weakened. When the concentration of stichopus oligopeptide increased to 20 μg/ml, there was no red band on the detection line. When the concentration of stichopus oligopeptide increased to 1000 μg/ml, there was no red band on the detection line and red band appeared on the quality control line. Therefore, it was judged that the detection sensitivity of the test strip was 20 μg/ml.

TABLE 1
Sensitivity detection results of colloidal
gold test strips in the present invention

| Stichopus | | | |
|---|---|---|---|
| oligopeptide/ | Parallel sample | | |
| μg/ml | 1 | 2 | 3 |
| 0 | − | − | − |
| 5 | − | − | − |
| 10 | − | − | − |
| 20 | + | + | + |
| 40 | + | + | + |
| 80 | + | + | + |
| 1000 | + | + | + |

Note:
“+” means positive,
“−” means negative

### Example 5

### Specificity Detection of Colloidal Gold Test Strip

Stichopus oligopeptide, soybean oligopeptide, marine fish oligopeptide and oyster oligopeptide were prepared respectively into 1 mg/ml samples, and they were detected according to the method described in Example 3. The results are as shown in Table 2. The results showed that there was no color in the detection line of stichopus oligopeptide samples, and while a red band appeared in the quality control line. Red bands appeared in the sample detection lines of soybean oligopeptide, marine fish oligopeptide and oyster oligopeptide, and red bands also appeared in the quality control line, which indicated that the test strip had good specificity.

TABLE 2
Specificity detection results of colloidal
gold test strips in the present invention

| | Stichopus | Soybean | Marine fish | Oyster |
|---|---|---|---|---|
| Sample | oligopeptide | oligopeptide | oligopeptide | oligopeptide |
| Result | + | − | − | − |

Note:
“+” means positive,
“−” means negative

It should be noted that the colloidal gold labeled monoclonal antibody in Example 2 may also be a mixture of a specific anti-KIVPGVPD monoclonal antibody and a specific anti-GRDGDQGPV monoclonal antibody; preferably, the mass ratio of the specific anti-KIVPGVPD monoclonal antibody to the specific anti-GRDGDQGPV monoclonal antibody is 1:1. One monoclonal antibody has the sequence of:

Light Chain (VL + CL):
(SEQ ID NO. 9)
DIVMTQSPSSLSASVGDRVTITCRASQSVSSYLAWYQQKPGKAPKLLI
YGSSNRASGVPDRFSGSGSGTDFTLTISSVEAEDVAVYYCKASQDVVLT
SASNRFSQQYYSTPWTFGQGTKVEIKRTVAAPSVFIFPPSDEQLKSGTA
SVVCLLNNFYPRDISVKWKIDGSERQNGVLNSWTDQDSKDSTYSMSSTL
TLTKADYEKHKVYACEVTHQGLSSPVTKSFNRGEC,
Heavy Chain (VH + CH):
(SEQ ID NO. 10)
EVQLVESGGGLVQPGGSLRLSCAASGFTFSSYAMSWVRQAPGKGLEWVS
AISDGGSYTYYADSVKGRFTISRDNAKNTLYLQMNSLRAEDTAVYYCAR
DGGDQLGGFDYWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPPCPAPELLGGPSVF
LFPPKPKDTLMISRTPEVTCVVVDVSHEDPEVKFNWYVDGVEVHNAKTK
PREEQYNSTYRVVSVLTVLHQDWLNGKEYKCKVSNKGLPSSIEKTISKA
KGQPREPQVYTLPPSQEEMTKNQVSLTCLVKGFYPSDIAVEWESNGQPE
NNYKTTPPMLDSDGSFFLYSKLTVDKSRWQQGNVFSCSVMHEALHNHYT
QKSLSLSPGK.

It should be noted that when the claims of the present invention involve numerical ranges, it should be understood that two endpoints of each numerical range and any value between the two endpoints can be selected. For the sake of avoiding elaboration, the present invention describes the preferred embodiments.

Although preferred embodiments of the present invention have been described, additional changes and modifications may be made to these embodiments once those skilled in the art have become aware of the basic inventive concepts. As such, that append claims are intended to be interpreted as including the preferred embodiments and all alterations and modification falling within the scope of the invention.

It will be apparent to those skilled in the art that various modifications and variations can be made in the present invention without departing from the spirit and scope of the invention. Thus, if these modifications and variations of the present invention fall within the scope of the claims of the present invention and their equivalent technologies, the present invention is also intended to include these modifications and variation

What is claimed is:

1. A monoclonal antibody against SEQ ID NO: 1 or SEQ ID NO: 3, wherein the monoclonal antibody comprises:
(a) a heavy chain variable region with amino acid sequence of SEQ ID NO: 5, and a light chain variable region with amino acid sequence of SEQ ID NO: 6;
or
(b) a heavy chain variable region with amino acid sequence of SEQ ID NO: 7, and a light chain variable region with amino acid sequence of SEQ ID NO: 8;
or
(c) a heavy chain variable region with amino acid sequence of SEQ ID NO: 10, and a light chain variable region with amino acid sequence of SEQ ID NO: 9.

2. A colloidal gold test strip comprising a sample pad, a gold label pad, an NC membrane, an absorbent pad and a PVC bottom plate, wherein the gold label pad is coated with the monoclonal antibody of claim 1.

3. The colloidal gold test strip of claim 2, wherein the NC membrane sequentially comprises a detection line and a quality control line according to the direction of sample chromatography, wherein the detection line is coated with a conjugate of peptide fragment SEQ ID NO: 1 or SEQ ID NO: 3 and bovine serum albumin; and the quality control line is coated with goat anti-mouse secondary antibody.


SEQUENCE LISTING (10 sequences)

SEQ ID NO: 1 - protein, 8 aa, Apostichopus japonicus
KIVPGVPD

SEQ ID NO: 2 - protein, 881 aa, Apostichopus japonicus
MGVFATGLDWELEAPNPIPGVPTDITIDASALFTPTSRNLEGDDLWRLSIFGSDNGRGSGPRVNENRQILDQDQASTTFTPGEPVDIDGITTEFDVGSVGCTDELTHVCVEFTRGDDPNPEYSFRTVDPLLGTEESIISCKELPCGAKAIFTELNADLTEPDVIIEDQPNTLVVDLTAVRHPTSTAVAGEDLWKVKSYFSPRDDGRGPKYDPKEEILTPAYKDTPLLYDQDLDFEGISIEADLSGVTCSQASYFCMEMEKDAAASIDYEFGTIPEQDPFVTCVDITDRCKVSIYHYGIVLFNPPRCGRHNLDFTREVPDFIPSGQPVPVTIDATIDMEPYSRGVSGACLWKVNMFGSRNNDGSGARQPLSSQILDSYEAAIPLSPGGPLYFENLQADFDVSQLGCGEIGYLCLEFTQCDTPNPDFSFEASDGGESIVKCQAENCRAVFIDDFTTNPIDIDLVEGRRSNPVNFQGTASSTDESGTVPSGFDLWTVSFWGSKKPNGKGEQQNPQEQVLDPYYASTPLYNPGDNIDMSRIFSNFDMDGLQCDDVPYLCAKFNRDPNSRPPFEMEAVPDERVLTSCFPVPEDSCKGVIATDLDFDIEFDKIVPGVPDDVTFDIDLSTLYTSAGADGNNLWRVGFFTSPDEIGSVKSNYDPQILTRSSSSTDLEPGEALKLDNVNTQVDLTGLGCEEDMKFVCLEFAKGQRANPDFKFEVSGGGDVLVKCIEQECEKPIVVTELEVQPYGTITVTEGNPTNRVLFSLTGVTDKDESGKAIGDDLWDLDTWGSTTEDCSGTKHNPKTQNNLQQSQMDRDVYEGQDVSLGAVDTEFDMSGLNCEEVKYFCAHLKKNAKAEPDFEFETKPDESAAIASFAVDCKGKGNL

SEQ ID NO: 3 - protein, 9 aa, Apostichopus japonicus
GRDGDQGPV

SEQ ID NO: 4 - protein, 1813 aa, Apostichopus japonicus
MISGVVLTDMNWDFTVQGPKKPNGSELLGINIQVDPARTSASVDGKRLWRAALFRSSSPNGDGPRVNSKMQILNKYQASRPLEGGGQTMRIDGVQAEFDMDEFAGCSNDYQYLCLEFTKNRKPKVNFRMTIEGGDRSIIKCKFQECMRGIELTDFEAEPGRPVELMEGKSENSVTFRLTATPSLQSGGIQGFDLWNLNVFGSLNPFGAGERINQVSEVLAPNQRSTGLFPPSIMNFGHVQHYFNMTGVECGDIMYLCAELVQGAEPNPEFQLNAIPDERVLIDCFNVHCLGIVIRETKLDLYNGGGLLDRDVDTNHLSFGVQILAGMESGDAVGDMLWRMEAYISNDYAGYGTRHVLKRQLLNIEQSGYDIHSGERLEFNNLATMLERDQLSCEEQLYLCVELMRGNDKIKFMGSRPGSLRDCEELNCAERPKPVVERFSFFLLHVVKFNLLHGLTGPKGPKGEPGQTPVIGSGDLIPGPPGPPGGRGGPGDRGPLGPKGNIGIPGPRGGSGADGVPGLPGPPGPPGPPGPSGQLTAEAQVSQQYKGPVMPAGLGYPQAMMAGEPGQRGPPGQAGLRGPAGLTGAPGEPGEQGPTGPVGLTGPAGSPGRPGSAGKDGANGNPGPAGPSGPPGPAGQNGFPGPSGPKGHRGYSGRAGQKGERGDDGERGNDGAAGSIGAPGPAGPMGSPGERGRTGPSGDTGPAGSDGAPGAQGPPGSAGIPGAPGVPGSSGPKGDAGSPGATGPQGQQGSRGERGPEGQQGQAGLQGAPGMNGNPGAKGASGAPGAQGPIGFSGLRGPNGAPGSQGPAGSKGDQGVAGAPGEQGEEGPTGLPGDVGPQGPPGANGASGERGPRGSTGPAGPQGPAGDRGQLGAPGSIGPAGAAGAKGETGARGGPGSPGSKGSRGEPGRNGDPGLPGTRGLSGNPGKQGKDGSPGPPGPPGENGRQGATGATGQRGMPGAQGLTGAPGAQGDRGGDGQVGPPGPAGPQGEVGDRGNPGPAGAPGSPGVAGERGAPGLQGPQGQQGPAGLNGPQGESGAPGDAGPQGETGAPGNPGDRGERGGPGDRGPPGPSGRPGGRGVSGRAGQDGATGAPGPKGEQGPPGGPGLGGQQGPRGPPGPSGSRGPPGEDGDEGRPGPTGPAGPRGSIGPIGPTGITGSPGEKGDQGSPGPAGAQGARGDAGARGANGPAGPQGFPGPAGRPGSPGARGETGPSGIRGETGPAGAAGATGDPGAQGPAGAVGAQGERGEAGNTGPQGPVGNPGIGGPQGNQGPPGPAGQTGPSGAPGAPGERGDPGVAGRDGDQGPVGAPGASGEKGDRGSDGDIGSAGPAGPPGPPGASGPLGIAGSMGPRGPPGAPGGPGEQGATGSPGSQGNRGSPGAIGAPGLLVQLVPMDKMVKMVVMVPKAQPDNVVRRETLVQPVTQDLKVAQDLRGLLDKMVPQESVVKLVPQAPLDPKDLLDNVDLLDQLAQLDLLVQLVNVVHLAHRVLKVIKDPLVSKGSPGDTGVPGPPGTAGERGSPGNRGAAGMDGATGPRGFDGPEGPRGPPGSEGRQGSQGPPGPPGPPGPPGTVQGFTYSQYPNQVVSGQDKGPSPYYYGDDANVDAKNKEPEILATLKDINNRFKALKQPSGDSKLHATRSCRNLFLDHPDKESGIYWVDPNLGCTDDAFQAFCDKTNKATCLTPETHLVTNATWYTGKPKRIYYSSMRGGEKFTYAEKSQLVFLRLLSISAKQSVTFYCRNVAANLEFLNSREDVMRDYQILENTCQSQSDSWGRVVLDYETDITDNLPFEDFSVGDLGADNQEFGLDMGKVCFA

SEQ ID NO: 5 - protein, 347 aa, synthetic construct
  REGION 1..347: note = Heavy Chain
QVQLVQSGAEVKKPGSSVKVSCKASGYAFTSYTMHWVKQAPGQGLEWIGYINPSRGYTNYNQKFKDKATLTTDKSSSTAYMQLSSLTSEDSAVYYCGYISGIYPGSGVRDGYDYWGQGTLVTVSSASTKGPSVFPLAPSSKSTSGGTAAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPASVSGSPGQSVEKTVAWKADSSPVKAGVETTTPSKQSNNKYAASSYLSLTPEQWKSHRSYSCQVTHEGSTVEKTVAPTECS

SEQ ID NO: 6 - protein, 230 aa, synthetic construct
  REGION 1..230: note = Light Chain
DIVMTQSPSFLSASVGDRVTITCRASQSVSSYLAWYQQKPGKAPKLLIYGSSNRASGVPDRFSGSGSGTDFTLTISSVEAEDVAVYYCKASQDVVLTSASNRFSQQYYSTPWTFGQGTKVEIKRTVAAPSVFIFPPSDEQLKSGTASVVCLLNNFYPRDISVKWKIDGSERQNGVLNSWTDQDSKDSTYSMSSTLTLTKADYEKHKVYACEVTHQGLSSPVTKSFNRGEC

SEQ ID NO: 7 - protein, 352 aa, synthetic construct
  REGION 1..352: note = Heavy Chain
QVQLVQSGAEVKKPGSSVKVSCKASGYAFTSYTMHWVKQAPGQGLEWIGYINPSRGYTNYNQKFKDKATLTTDKSSSTAYMQLSSLTSEDSAVYYCRASQDISNYLNYASRLSGRYFDYWGQGTLVTVSSASTKGPSVFPLAPSSKSTSGGTAAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPASVSGSPGQSVEKTVAWKADSSPVKAGVETTTPSKQSNNKYAASSYLSLTPEQWKSHRSYSCQVTHEGSTVEKTVAPTECS

SEQ ID NO: 8 - protein, 230 aa, synthetic construct; identical to SEQ ID NO: 6
  REGION 1..230: note = Light Chain

SEQ ID NO: 9 - protein, 230 aa, synthetic construct
  REGION 1..230: note = Light Chain
DIVMTQSPSSLSASVGDRVTITCRASQSVSSYLAWYQQKPGKAPKLLIYGSSNRASGVPDRFSGSGSGTDFTLTISSVEAEDVAVYYCKASQDVVLTSASNRFSQQYYSTPWTFGQGTKVEIKRTVAAPSVFIFPPSDEQLKSGTASVVCLLNNFYPRDISVKWKIDGSERQNGVLNSWTDQDSKDSTYSMSSTLTLTKADYEKHKVYACEVTHQGLSSPVTKSFNRGEC

SEQ ID NO: 10 - protein, 451 aa, synthetic construct
  REGION 1..451: note = Heavy Chain
EVQLVESGGGLVQPGGSLRLSCAASGFTFSSYAMSWVRQAPGKGLEWVSAISDGGSYTYYADSVKGRFTISRDNAKNTLYLQMNSLRAEDTAVYYCARDGGDQLGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPMLDSDGSFFLYSKLTVDKSRWQQGNVFSCSVMHEALHNHYTQKSLSLSPGK